(19) United States
(12) Patent Application Publication
(10) Pub. No.: US 2011/0028913 A1
(43) Pub. Date: Feb. 3, 2011

(54) GASKET, AND ITS UTILIZATION

(75) Inventors: Yasuhiro Muramatsu, Shizuoka (JP); Hiroaki Mizuno, Toyama (JP); Junya Okazaki, Gifu (JP); Yusuke Ishita, Gifu (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Taiyo Pharmaceutical Industry Co., Ltd., Nagoya-shi (JP)

(21) Appl. No.: 12/738,160

(22) PCT Filed: Oct. 15, 2008
(86) PCT No.: PCT/JP08/68620

§ 371 Date: Oct. 25, 2010

(30) Foreign Application Priority Data
Oct. 17, 2007 (JP) 2007-270616

(51) Int. Cl. A61M 5/315; F16J 15/16

(52) U.S. Cl. 604/222; 277/630

(57) ABSTRACT

The present invention is intended to provide a gasket which is able to be mounted on a plunger with a loosening preventing mechanism and, more specifically, a gasket including loosening preventing means provided for the plunger having a mechanism configured to prevent a joint portion between the plunger and the gasket from loosening, and a prefilled syringe preparation utilizing this gasket. The gasket is mountable on the plunger having a projecting portion in a groove of a screw portion for screwing the gasket thereon including a depressed portion with which the projecting portion is engageable when being screwed on the plunger provided on an inner periphery of the gasket.

### TECHNICAL FIELD

The present invention relates to a gasket which is able to be mounted on a plunger with a loosening preventing mechanism and, more specifically, to a gasket including loosening preventing means provided for the plunger having a mechanism configured to prevent a joint portion between the plunger and the gasket from loosening, and a syringe or a prefilled syringe preparation utilizing this gasket.

### BACKGROUND ART

A syringe of so-called a prefilled type including medicine filled in advance is shipped and distributed in a state of being filled with drug solution in an interior of a barrel and sealing the barrel with a plunger having a gasket joined to a distal end portion thereof.

However, this syringe has a problem such that there is a case where a joint between the plunger and the gasket is disconnected and only the plunger comes apart due to an impact such as vibrations from the outside received during distribution or the like. In particular, in the prefilled syringe preparation used for pharmaceutical preparations, an attention is needed for a problem of deterioration of workability at the time of administering a dose or difficulty of adequate infusion of drug solution due to coming apart or loosening of the plunger.

In order to prevent such the problem, in the related art, an attempt is made to bond the plunger with the gasket or mold the same integrally with each other to prevent the plunger from coming apart.

However, since integrating the plunger and the gasket as described above causes obstacles when sorting them for discarding after the usage and increases the number of steps of manufacture, it is not a desirable method in terms of cost.

Accordingly, there is developed a plunger screw latching mechanism having a projecting portion at the distal end portion of the plunger and being configured to prevent a screw at a joint portion from coming apart due to the vibrations from the outside although joining the plunger and the gasket in a screw-in system (see Patent Document 1). However, in this preventing mechanism, since the projecting portion is formed at the distal end portion of the plunger, when the screwing fixation between the plunger and the gasket is loosened to some extent and hence there is a gap at a joint surface between the plunger and the gasket even if only slightly, there is a probability of loosening of engagement between a proximal end of the gasket and the projecting portion. In addition, in the prefilled syringe preparation in which the drug solution is to be filled therein, the drug solution enters the gap during the manufacturing process, and hence it is not necessarily a preferable mode for being used for the pharmaceutical preparations.

Furthermore, in this plunger screw latching mechanism, although the engagement between the proximal end of the gasket and the projecting portion is achieved only when the screwing is completely done so as not to generate the gap at the joint surface between the plunger and the gasket, it is difficult to confirm the state of engagement, and hence there is a demand for a syringe in which the loosening of the joint between the plunger and the gasket is prevented more easily and reliably.

Patent Document 1: JP-A-2004-215896

### DISCLOSURE OF INVENTION

### Problems to be Solved by the Invention

Accordingly, the present invention is intended to provide a gasket which overcomes defects of the syringe in the related art and is configured to allow reliable prevention of loosening of a joint between a plunger and a gasket simply by screwing the plunger into the gasket.

### Means for Solving the Problems

The present invention is intended to solve the above-described problems, so that there is provided a gasket mountable on a plunger having a projecting portion on a screw portion for screwing the gasket thereon including a depressed portion with which the projecting portion is engageable when being screwed on the plunger provided on an inner periphery of the gasket.

The present invention is also intended to solve the above-described problems, so that there is provided a gasket mountable on a plunger having a depressed portion on a screw portion for screwing the gasket thereon including a projecting portion with which the depressed portion is engageable when being screwed on the plunger provided on an inner periphery of the gasket.

### ADVANTAGES OF THE INVENTION

Since the gasket according to the present invention includes the depressed portion or the projecting portion which are engageable respectively with the projecting portion or the depressed portion provided on the screw portion of the plunger for preventing loosening, by screwing and mounting the gasket on the plunger, the projecting portion and the depressed portion engage with each other and, consequently, the joint between the plunger and the gasket do not loosen even when an impact such as the vibrations is applied from the outside.

Therefore, the syringe using the gasket according to the present invention has advantages such that the loosening of the joint between the plunger and the gasket are able to be prevented easily and reliably and also manufacturing and discarding are easy.

### BEST MODES FOR CARRYING OUT THE INVENTION

Referring now to drawings, embodiments of the present invention are described further in detail. However, the present invention is not limited to these embodiments.

FIG. 1 is a perspective view of a gasket according to the present invention, FIG. 2 is a cross-sectional view of the gasket according to the present invention, FIG. 3 is a front view of a plunger with a loosening preventing mechanism to which the gasket according to the present invention is screwed, FIG. 4 is a lateral cross-sectional view taken along a direction A-A′ of a plunger screw portion with the loosening preventing mechanism, and FIG. 5 is a drawing showing another embodiment of the plunger with the loosening preventing mechanism. In the drawings, reference numeral 1 designates a gasket, reference numeral 2 designates a plunger screw portion, reference numeral 3 designates a plunger body portion, reference numeral 11 designates a screw thread, reference numeral 12 designates a depressed portion, reference numeral 13 designates a screw groove, reference numeral 21 designates a screw groove, reference numeral 22 designates a projecting portion, and reference numeral 23 designates a screw thread, respectively.

As shown in FIG. 1 and FIG. 2, the gasket 1 according to the present invention is formed into a cap shape which allows mounting to a distal end of the plunger body portion 3, and is formed with the screw thread 11 on an inner periphery thereof so as to be screwed into the groove portion 21 of the plunger screw portion 2 provided at a distal end of the plunger body portion 3.

As shown in FIG. 3, the plunger screw portion 2 is formed with the projecting portions 22 in the groove portion 21. The projecting portions 22 are each formed into a wedge shape having a distal portion in the direction of rotation of the plunger when screwing the plunger into the gasket in plan view as shown in FIG. 4.

Here, the projecting portions 22 will be described further in detail. In this embodiment, two pairs of the projecting portions 22 are provided in axial symmetry in the groove portion 21 rather on the side of the joint portion with respect to the plunger 3 from a substantially midsection of the height of the screw portion 2 at four positions in total. However, the positions and the number of provisions are not limited thereto, and what is essential is not to prevent the screwing of the gasket on the plunger, and to ensure the engagement of the projecting portions 22 with the gasket 1 after the screwing. Although the provision of the projecting portion 22 may be only at one position, it is preferable to provide the same at a plurality of positions in order to further ensure the engagement between the projecting portions 22 and the gasket 1. Although the positions, sizes, and heights of the projecting portions 22 in the screw portion 2 are arbitrary, it is preferable to provide the same rather on the side of the joint portion with respect to the plunger from a position in the vicinity of the midsection of the height of the screw portion for the purpose of preventing the loosening of the same at the time of joining the gasket and the plunger and also from a point of view that screwing of the gasket on the plunger and slidability when using the syringe are preferably prevented from being hindered, so that the height thereof in the screw groove preferably does not exceed an end portion of an apex of the screw thread. In this embodiment, although the projecting portions 22 are formed into the wedge shape, they may be formed into any shape basically as long as the gasket is not loosened after screwed in once. In addition, when providing the plurality of projections, the positional relationship among the respective projections is arbitrary. However, it is preferable to be equidistant with respect to a peripheral surface of the screw. Although the plunger 1 and the projecting portions 22 maybe formed by selecting different materials and bonding to each other after the formation of the screw portion 2, it is more preferable to select the same material from polypropylene, polyethylene terephthalate, polystyrene, ABS or the like, and obtain the same by integral molding.

The gasket 1 is formed with the depressed portion 12 by notching part of the screw thread 11 so as to achieve engagement of the projecting portions 22 described above when the gasket 1 is screwed on the plunger 3. The position where the depressed portion 12 is provided is preferably set so that the projecting portions 22 engages therewith in a state where the screw is completely tightened after having screwed the gasket 1 on the plunger 3 because the fact that the screw is reliably tightened can be confirmed when mounting the gasket 1 on the plunger 3. Although the shape and the size thereof are arbitrary as long as engagement of the projecting portions 22 is ensured according to the shape and the size of the projecting portions 22, it is preferable to form one side of the projecting portions 22 in the direction of travel obliquely because smooth screwing of the projecting portions 22 is ensured. Specifically, although the depressed portion 12 in this embodiment is formed into a substantially rectangular shape in plan view by notching the screw thread 11 from an apex portion toward a bottom portion thereof at a substantially right angle, if one side of the projecting portions 22 in the direction of travel is formed obliquely so as to widen an entrance of the depressed portion 12, the friction at the time of inserting the projecting portions 22 into the depressed portion 12 is reduced interdependently with the distal end side of the wedge shape of the projecting portions 22. In contrast, since other sides are right angles, engagement with the rear end side of the wedge shape of the projecting portions 22 is ensured. The material of the gasket 1 may be any material as long as it has resiliency and does not have critical effect on the quality of the drug solution as contents of a syringe, and may be selected as needed from natural rubber, isoprene rubber, butadiene rubber, butyl rubber, thermal plastic elastomer, and so on.

A mechanism for preventing the loosening of the joint between the gasket 1 and the plunger 3 by screwing the former on the latter, is described in detail below.

First of all, when the gasket 1 is screwed into the screw portion 2 of the plunger 3, since the projecting portions 22 is not provided on the side of an upper portion of the plunger screw portion 2, insertion is achieved without friction in the initial stage. When screwing is proceeded to a position where the screw thread 11 of the gasket 1 comes into contact with the projecting portions 22, the projecting portions 22 is inserted so as to push and widen the gasket 1 having resiliency from inside while being in abutment with the screw thread 11. Then, when screwing is proceeded until when the projecting portions 22 finally reaches the position of the depressed portion 12 as is, the projecting portions 22 is accommodated in the depressed portion 12, and the depressed portion 12 and the projecting portions 22 formed into the wedge shapes engage with each other, so that the rotation in the opposite direction is restrained.

In this manner, by the provision of the depressed portion 12 for being engaged with the projecting portions 22 on the inner periphery of the gasket 1, engagement of the gasket 1 and the plunger 3 is ensured and hence the loosening therebetween is effectively prevented. However, since the projecting portions 22 are accommodated in the depressed portion 12 and hence there is no probability of deformation such as pressing the screw thread 11 and causing the corresponding portion of the gasket 1 to swell, so that smooth sliding of the syringe in the plunger is not hindered.

FIG. 5 shows another embodiment of the plunger according to the present invention, in which the position where the projecting portions 22 are provided is shifted by 90 degrees counterclockwise in plan view in comparison with the one shown in FIG. 3, that is, it is formed near the midsection of the screw portion 2 in comparison with the configuration in FIG. 3.

Although the gasket 1 having the depressed portion 12 which is able to be mounted to the plunger 3 having the projecting portions 22 has been described in the above-mentioned embodiment, in contrast, it is also possible to form the depressed portion on the plunger 3 and the projecting portion on the gasket 1 correspondingly. In such a case, the depressed portion is formed by notching part of the screw thread 23 of the plunger 3 and the projecting portion which is engageable with the depressed portion is formed in the screw groove 13 of the gasket 1. The same conditions as described in the above-mentioned embodiment may be employed as the shape, the size, the positions, and the number of the projecting portions and the depressed portions, and the same effects as the above-mentioned embodiment are achieved.

### EXAMPLES

Subsequently, the present invention will be described further in detail with examples.

### Example 1

The gasket according to the present invention and a gasket having no depressed portion formed on the screw thread in the gasket were mounted on the plungers having the projecting portions respectively on the screw portions, a load was applied in a direction to tighten the screw in the state in which the depressed portion of the gasket and the projecting portion of the plunger are engaged with each other, and torque resistances were measured. A result as shown in Table 1 was obtained. This test was conducted using a configuration in which the positions of the depressed portions were formed corresponding to the positions of the projecting portions shown in FIG. 3 (shown as “projecting portion-1”), and a configuration in which the positions of the depressed portions were formed corresponding to the positions of the projecting portions shown in FIG. 5 (shown as “projecting portion-2”).

TABLE 1
Unit: Nm

| | Without Depressed Portion | | With Depressed Portions | |
|---|---|---|---|---|
| | projecting | projecting | projecting | projecting |
| | portion-1 | portion-2 | portion-1 | portion-2 |
| 1 | 0.013 | 0.018 | 0.015 | 0.035 |
| 2 | 0.01 | 0.017 | 0.016 | 0.026 |
| 3 | 0.01 | 0.015 | 0.016 | 0.028 |
| 4 | 0.011 | 0.016 | 0.019 | 0.032 |
| 5 | 0.012 | 0.018 | 0.02 | 0.029 |
| AV | 0.0112 | 0.0168 | 0.0172 | 0.03 |
| SD | 0.0013 | 0.0013 | 0.0022 | 0.0035 |
| MAX | 0.013 | 0.018 | 0.02 | 0.035 |
| MINI | 0.01 | 0.015 | 0.015 | 0.026 |

### Example 2

The gasket according to the present invention and a gasket having no depressed portion formed on the screw thread in the gasket were mounted on the plungers having the projecting portions respectively on the screw portions, the load was applied in a direction to loosen the screw in the state in which the depressed portion of the gasket and the projecting portion of the plunger are engaged with each other, and the torque resistances were measured. A result as shown in Table 2 was obtained. This test was conducted using a configuration in which the positions of the depressed portions were formed corresponding to the positions of the projecting portions shown in FIG. 3 (shown as “projecting portion-1”), and a configuration in which the positions of the depressed portions were formed corresponding to the positions of the projecting portions shown in FIG. 5 (shown as “projecting portion-2”).

TABLE 2
Unit: Nm

| | without Depressed Portion | | With Depressed Portion | |
|---|---|---|---|---|
| | projecting | projecting | projecting | projecting |
| | portion-1 | portion-2 | portion-1 | portion-2 |
| 1 | 0.015 | 0.025 | 0.021 | 0.045 |
| 2 | 0.014 | 0.024 | 0.017 | 0.046 |
| 3 | 0.016 | 0.025 | 0.024 | 0.04 |
| 4 | 0.012 | 0.023 | 0.023 | 0.043 |
| 5 | 0.013 | 0.029 | 0.024 | 0.046 |
| AV | 0.014 | 0.0252 | 0.0218 | 0.044 |
| SD | 0.0016 | 0.0023 | 0.0029 | 0.0025 |
| MAX | 0.016 | 0.029 | 0.024 | 0.046 |
| MINI | 0.012 | 0.023 | 0.017 | 0.04 |

As is understood from the result of the test shown above, according to the gasket in the present invention, it was found that the resistance of the one formed with the depressed portion in the gasket was higher, that is, the gasket was firmly engaged and hence could not easily be disconnected in comparison with the one not formed with the depressed portion, and the resistance was higher, that is, the gasket was firmly engaged and hence could not easily be disconnected in the configuration in which the positions of the depressed portion of the gasket and the projecting portion of the plunger were rather closer to the midsection of the screw portion (projecting portion-2) with respect to the proximal end of the screw portion.

### Example 3

Three configurations, namely, a configuration in which the gasket according to the present invention was mounted on the plunger having the projecting portion at the position shown in FIG. 5, a configuration in which the gasket which was not formed with the depressed portion on the screw thread was mounted on the plunger having the projecting portion at the position shown in FIG. 5, and a configuration in which the gasket which was not formed with the depressed portion on the screw thread was mounted on the plunger which was not formed with the projecting portion were used to measure the respective effects on the low-speed slidability. Specifically, the sliding resistance of the plunger in the cylinder was measured while maintaining an internal pressure of the syringe at 338 mmHg, and a speed of infusion of 0.05 mm/min (1 mL/h).

From the test described above, the gasket according to the present invention was found that the sliding resistance was little changed in comparison with the combination of the gasket which was not formed with the depressed portion on the screw thread and the plunger which was not formed with the projecting portion, that is, a general combination of the gasket and the plunger having no loosening preventing mechanism of the present invention even when the plunger was slid at a low speed in the cylinder, so that the gasket according to the present invention slides smoothly in the syringe. Also, the gasket according to the present invention was found to have less sliding resistance, and hence to slide more smoothly in the syringe in comparison with the combination of the gasket which was not formed with the depressed portion on the screw thread and the plunger which was formed with the projecting portions.

### BRIEF DESCRIPTION OF THE DRAWINGS

FIG. 1 is a perspective view of a gasket according to the present invention.

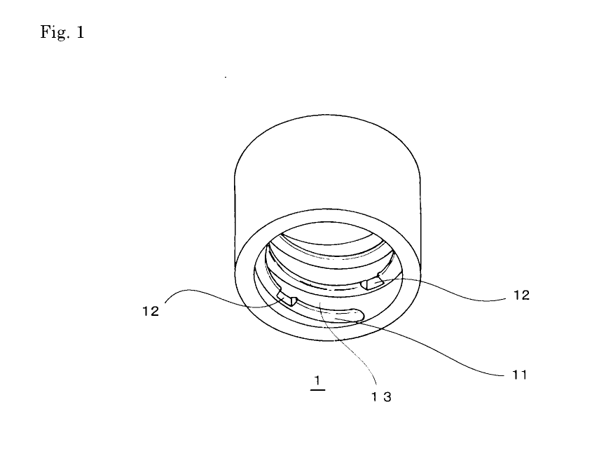

FIG. 2 is a cross-sectional view of the gasket according to the present invention.

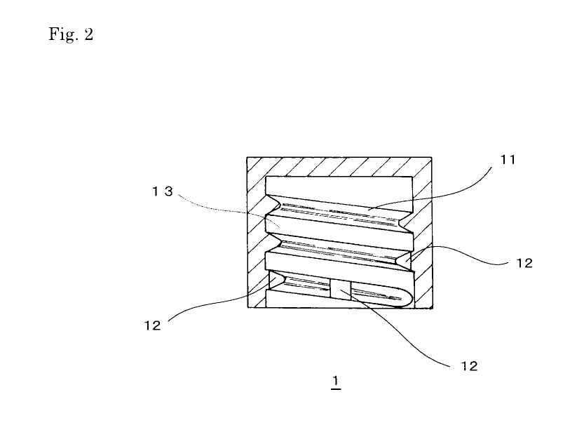

FIG. 3 is a front view of a plunger to which the gasket according to the present invention is joined.

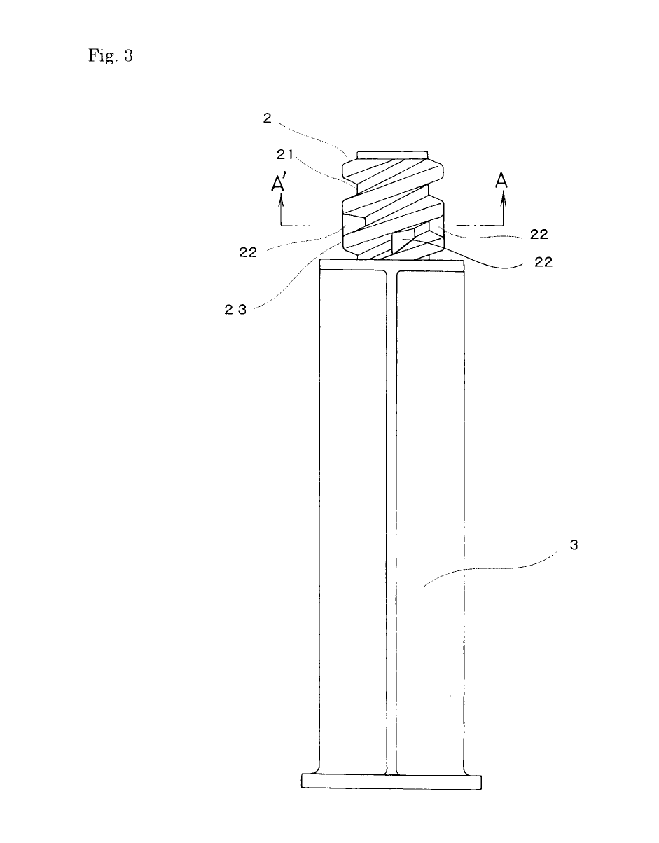

FIG. 4 is a lateral cross-sectional view taken along a direction A-A′ of a plunger screw portion according to the present invention.

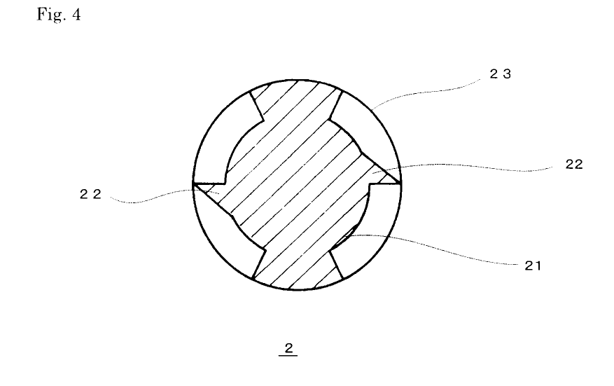

FIG. 5 is another embodiment of the plunger according to the present invention.

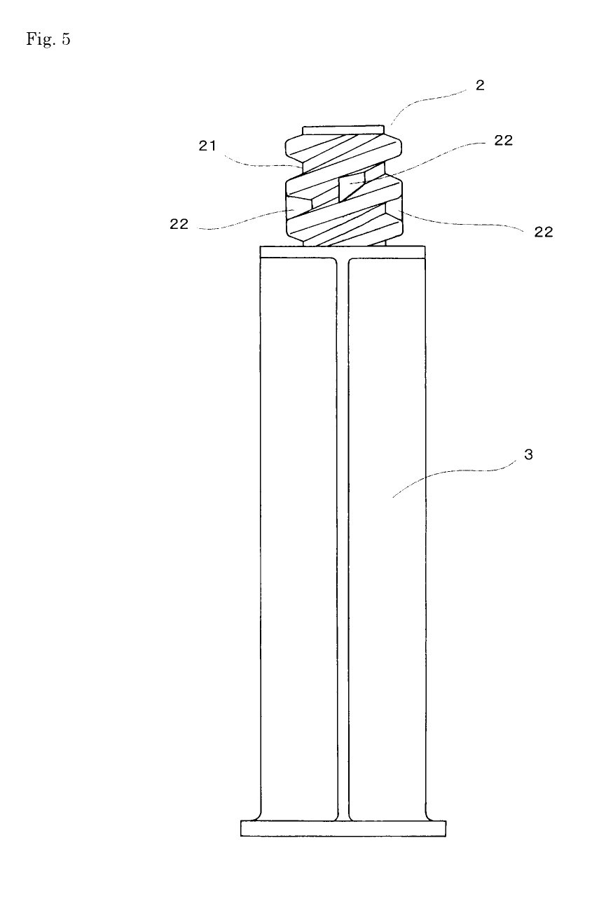

### REFERENCE NUMERALS

1 gasket
2 plunger screw portion
3 plunger
11 screw thread
12 depressed portion
13 groove portion
21 groove portion
22 projecting portion
23 screw thread

1. A gasket mountable on a plunger having a projecting portion on a screw portion for screwing the gasket thereon, comprising: a depressed portion with which the projecting portion is engageable when being screwed on the plunger provided on an inner periphery of the gasket.

2. The gasket according to claim 1, wherein the depressed portion is provided on part of a screw thread formed on the inner periphery of the gasket so as to be able to be screwed into the screw portion.

3. The gasket according to any one of claim 1 or claim 2, wherein the depressed portion is provided at a position corresponding to the position of the projecting portion when the gasket is screwed and mounted on the plunger.

4. The gasket according to any one of claim 1 or claim 2, wherein at least two or more the depressed portions are provided.

5. The gasket according to any one of claim 1 or claim 2, wherein when the plurality of depressed portions are provided, the depressed portions are provided equidistantly on the screw thread.

6. A gasket mountable on a plunger having a depressed portion on a screw portion for screwing the gasket thereon, comprising: a projecting portion with which the depressed portion is engageable when being screwed on the plunger provided on an inner periphery of the gasket.

7. The gasket according to claim 6, wherein the projecting portion is provided on part of a screw groove formed on the inner periphery of the gasket so as to be able to be screwed into the screw portion.

8. The gasket according to any one of claim 6 or claim 7, wherein the projecting portion is provided at a position corresponding to the position of the depressed portion when the gasket is screwed and mounted on the plunger.

9. The gasket according to any one of claim 6 or claim 7, wherein at least two or more the projecting portions are provided.

10. The gasket according to any one of claim 6 or claim 7, wherein when the plurality of projecting portions are provided, the projecting portions are provided equidistantly on the screw groove.

11. A syringe using the gasket according to claim 1.

12. a prefilled syringe preparation using the gasket according to claim 1.
